(19) United States
(12) Patent Application Publication
(10) Pub. No.: US 2016/0011138 A1
(43) Pub. Date: Jan. 14, 2016

(54) GLUCOMETER SYSTEM AND METHOD

(71) Applicants: Cindy Mason, Burleson, TX (US); Justin Lindsay, Burleson, TX (US)

(72) Inventors: Cindy Mason, Burleson, TX (US); Justin Lindsay, Burleson, TX (US)

(21) Appl. No.: 14/327,563

(22) Filed: Jul. 10, 2014

(51) Int. Cl. G01N 27/327; G01N 33/49

(52) U.S. Cl. CPC G01N 27/3272; G01N 33/49

(57) ABSTRACT

The present application relates to an interactive glucose system that permits a user to customize and efficiently manage glucose levels. The system includes a glucose reader configured to interact with a test strip to generate a voltage representative of a glucose level within a blood sample. The voltage is transmitted to an electronic device to generate a glucose reading. The electronic device is configured to store and display data related to the glucose reading. The electronic device and the reader are independent and detachable from one another. The system permits for the use of multiple users to operate and test glucose levels from a single device.

### BACKGROUND

1. Field of the Invention

The present application relates generally to glucometers, and in particular, to an interactive glucometer for mobile electronic devices.

2. Description of Related Art

A glucometer, or glucose meter, is an electronic device that determines the approximate concentration of glucose within the blood via a small blood sample. Blood samples are typically provided through a strip of glucose paper inserted into the meter. The meters are usually individual battery operated handheld devices that include a measurement engine, a processing unit, and a control/display system. The measurement engine includes the necessary sensors and modules to process the blood and relay a voltage to the processing unit. The processing unit receives the voltage and interprets the data to determine a glucose level. The processing unit is in communication with the display system to provide a visual indication of the level to a user. Typically the glucometer is an all-inclusive unit having each of these functions.

A single unit has some disadvantages. First, users are generally encouraged not to share glucometers in an effort to not contaminate or mix the blood. The processing unit and display may not ever come into contact with blood but still may not be shared since it is all one unit. Secondly, the size of glucometers can become a hindrance. Tests may be needed several times a day which may necessitate the user to carry this separate device around with them.

Additionally, a glucometer is known to be capable of communicating with one of a number of separate individual devices. Typically the results are processed by the glucometer and then transmitted to another device. This configuration still requires the carrying of an entire glucose meter. Another disadvantage of present glucometers is an inability of the clock to accurately represent the time of past test results. Incorrect times may result from static electricity, loss of power, or the failure to adjust during travel through multiple time zones.

A more convenient and adaptable interactive glucometer system is needed. Considerable shortcomings remain.

### DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a representation of an interactive glucometer system according to the preferred embodiment of the present application;

FIG. 2 is an exemplary embodiment of the interactive glucometer system of FIG. 1;

FIG. 3 is a schematic of a processing system used within the interactive glucometer system of FIG. 1; and

FIG. 4 is a flowchart of the operation and programing of the processing system used in the interactive glucometer system of FIG. 1.

While the system and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE PREFERRED EMBODIMENT

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the device described herein may be oriented in any desired direction.

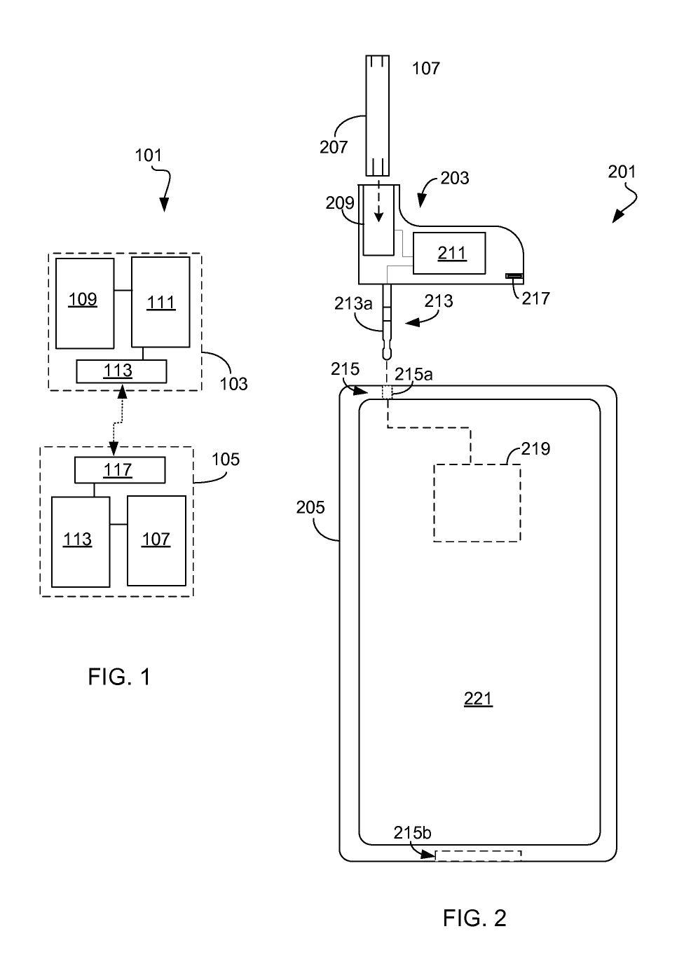

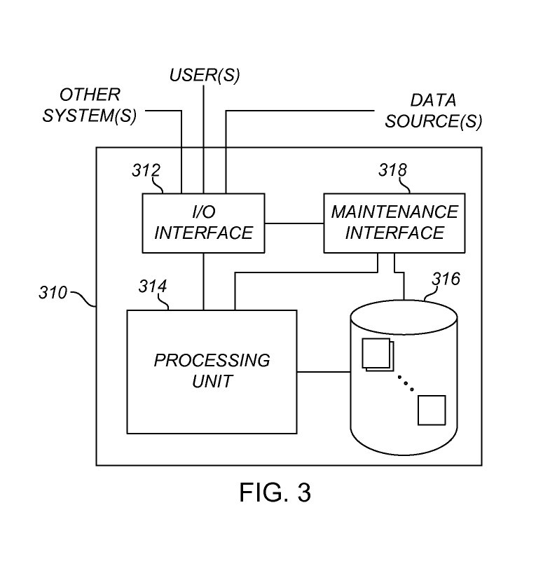

Referring now to FIGS. 1 and 2 in the drawings, an interactive glucometer system 101 is illustrated. FIG. 1 illustrates system 101 in a basic form. FIG. 2 illustrates an exemplary embodiment of the system of FIG. 1. In the basic form, system 101 includes two separate devices. Each device performs a certain purpose in determining the glucose level of a user. The first device 103 (glucose reader) specifically interacts with a testing strip within a port 109 and includes a measurement engine 111 having one or more amplifiers, converters, and modules necessary to process a blood sample located on the testing strip in order to produce a voltage representative of a glucose level. Second device 105 includes a processing unit or microprocessor configured to process the voltage generated by device 103 and calculate a glucose reading. Processing unit 113 is in communication with display 107 for representing the glucose reading to the user. Device 105 is configured to provide power and command data to device 103 to process the blood sample and generate the voltage.

As seen in FIG. 1, device 103 and 105 are two separate devices. This configuration separates port 109 and measurement engine 111 from the processing unit 113 and display 107. Device 103 and 105 each include a connector portion 115 and 117 adapted to permit communication between processing unit 113 and measurement engine 111. Because the connection between device 103 and 105 is detachable, device 105 is programmed to include the ability to handle one or more users. Only device 103 need remain with a particular user. Device 103 is designed to be compact and inconspicuous. The small relative size of device 103 being a result of the need to not have processing unit 113 and display 117. Only testing port 109 and measurement engine 111 are needed within device 103

An advantage of device 105 is the ability of a user to manage and review data related to previous tests without the need to have device 103 coupled to connector portion 117. A user may transmit, manage, and adjust the data at any time while device 105 is disconnected with device 103. Data is stored on device 105 independent from device 103.

Referring now in particular to FIG. 2. As stated previously, FIG. 2 illustrates an exemplary embodiment of each device 103 and 105 within FIG. 1. Therefore, interactive glucometer system 201 of FIG. 2 is similar in form and function to system 101, including the individual components of FIG. 1. System 201 includes a glucose reader 203 and an electronic device 205. Reader 203 is configured to selectively couple to device 205.

Reader 203 is configured to process a blood sample located on testing strip 207 and generate a representative voltage reading that correlates to a set glucose level within the blood sample. Port 209 is configured to accept testing strip 207. Port 209 holds testing strip 207 in place during testing of the blood sample. Testing strip 207 is detachable when testing is complete. Multiple testing strips may be used within port 209 on an individual basis as testing is required. As a result of testing blood and the desire to not contaminate future testing, reader 203 is configured to operate with a single user.

Reader 203 further includes a measurement engine 211. Engine 211 includes one or more amplifiers, converters, and modules necessary to process the blood sample located on testing strip 207 in order to produce the representative voltage. In way of example, engine 211 may include a converter to either convert A/C current to D/C current or D/C current to A/C current. Additionally, a trans-impedance amplifier used to convert the current inputs to a voltage may also be used. Reader 203 is configured to generate the voltage and transmit such voltage to device 205 for processing in order to obtain a glucose reading. It is recognized that some embodiments of reader 203 may further include a lancing device 217 configured to prick the skin of a user in order to draw a fresh blood sample. The pricking of the user pierces the skin. The blood is then placed on testing strip 207 for analysis.

Electronic device 205 is similar in form and function to device 105 in FIG. 1. Device 205 may be a personal computer, a portable computer such as a laptop or handleld device (e.g., a multimedia and internet enabled mobile phone or tablet). Such devices are in common use within the general public. Many such devices are already carried and can be easily operated. Device 205 includes programs necessary to store, process, operate, and transmit information related to each testing of glucose levels for a user.

Device 205 is configured to receive the voltage reading from device 203 and process the voltage reading to determine a glucose reading. Processing is performed within a processing unit 219, such as a microprocessor. Once the blood sample is analyzed by engine 211 and the voltage is processed by the processing unit 219, processing unit 219 transmits the representative data to display 221 for viewing by the user. Display is configured to present data in at least one of a graphical or numerical form. Additionally, display 221 and device 205 is configured to audibly display or convey results and historical data for a user.

Referring now also to FIG. 3 in the drawings, an exemplary illustration of the electrical components of device 205. FIG. 3 illustrates an exemplary processing system 310 used in device 205 for processing and regulating the testing of glucose levels from a blood sample. The processing system 301 includes an input/output (I/O) interface 312, processing unit 314 (processing unit 219 similar in form and function to that of unit 314), a database 316, and a maintenance interface 318. Alternative embodiments can combine or distribute the input/output (I/O) interface 312, processing unit 314, database 316, and maintenance interface 318 as desired. As stated previously, embodiments of the processing system 310 can include one or more computers that include one or more processors (e.g., processing unit 219) and memories configured for performing tasks described herein below. This can include, for example, a computer or handheld device having a central processing unit (CPU) and non-volatile memory that stores software instructions for instructing the CPU to perform at least some of the tasks described herein. This can also include, for example, two or more computers that are in communication via a computer network, where one or more of the computers includes a CPU and non-volatile memory, and one or more of the computer's non-volatile memory stores software instructions for instructing any of the CPU(s) to perform any of the tasks described herein. Thus, while the exemplary embodiment is described in terms of a discrete machine, it should be appreciated that this description is non-limiting, and that the present description applies equally to numerous other arrangements involving one or more machines performing tasks distributed in any way among the one or more machines. It should also be appreciated that such machines need not be dedicated to performing tasks described herein, but instead can be multi-purpose machines, for example computer workstations, smart phones, and tablets; that are suitable for also performing other tasks. Furthermore the computers may use transitory and non-transitory forms of computer-readable media. Non-transitory computer-readable media is to be interpreted to comprise all computer-readable media, with the sole exception of being a transitory, propagating signal.

The I/O interface 312 provides a communication link between external users, systems, and data sources and components of the processing system 310. This communication link may support the ability to transmit and receive data via fax, the internet, email, and instant messaging for example. The I/O interface 312 can be configured for allowing one or more users to input information to the processing system 310 via any known input device. Examples can include a keyboard, mouse, touch screen, microphone, and/or any other desired input device. The I/O interface 312 can be configured for allowing one or more users to receive information output from the processing system 310 via any known output device. Examples can include a display monitor, a printer, a speaker, and/or any other desired output device. The I/O interface 312 can be configured for allowing other systems to communicate with the processing system 310. For example, the I/O interface 312 can allow one or more remote computer(s) to access information, input information, and/or remotely instruct the processing system 310 to perform one or more of the tasks described herein. The I/O interface 312 can be configured for allowing communication with one or more remote data sources. For example, the I/O interface 312 can allow one or more remote data source(s) to access information, input information, and/or remotely instruct the processing system 310 to perform one or more of the tasks described herein (e.g., provide alerts and notifications to a user).

The database 316 provides persistent data storage for processing system 310. While the term “database” is primarily used, a memory or other suitable data storage arrangement may provide the functionality of the database 316. In alternative embodiments, the database 316 can be integral to or separate from the processing system 310 and can operate on one or more computers. The database 316 preferably provides non-volatile data storage for any information suitable to support the operation of the processing system 310, including various types of data discussed herein.

The maintenance interface 318 is configured to allow users to maintain desired operation of the processing system 310. In some embodiments, the maintenance interface 318 can be configured to allow for reviewing and/or revising the data stored in the database 316 and/or performing any suitable administrative tasks commonly associated with database management. This can include, for example, updating database management software, revising security settings, and/or performing data backup operations. In some embodiments, the maintenance interface 318 can be configured to allow for maintenance of the processing unit 314 and/or the I/O interface 312. This can include, for example, software updates and/or administrative tasks such as security management and/or adjustment of certain tolerance settings. The processing unit 314 can include various combinations of one or more processors, memories, and software components.

Referring back to FIG. 2, device 205 communicates with reader 203 through connection port 213 and connection port 215. As seen in FIG. 2, connection ports 213, 215 are illustrated as operating through a headphone jack 213a and corresponding receiving port 215a. In alternative embodiments, port 213 may be modified so as to operate with pin/charging and docking connector 215b of device 205. Other embodiments may rely on other wireless communications between reader 203 and device 205, such as: Bluetooth and infrared. Jack 213a and corresponding port 215a are illustrated for exemplary use only in this application.

Communication between reader 203 and device 205 operates bi-directionally. Device 205 is a powered device with a battery. An advantage of using connectors 213, 215 that physically connect is that power may be provided by device 205 to reader 203 in order to perform the necessary functions within engine 211 to generate the representative voltage. This allows reader 203 to be manufactured without the additional cost of a battery. Additionally, command data is configured to be transmitted by device 205 to reader 203 through connectors 213, 215 in order to regulate the analysis of the blood sample. For example, the command data may start and/or stop the analysis of the blood sample by reader 203, or the processing of the voltage by processing unit 219 This bi-directional communication offers the advantage that reader 203 does not need to maintain an extra power source. Furthermore, the cost to produce and purchase reader 203 is minimized. Secondly, the user may control the entire testing process from device 205.

Although device 205 has been described as being in use with a single user, it is understood that multiple users may not only access or transmit data from a remote location, but that device 205 is configured to handle multiple users specific to device 205 in particular. Processing unit 219 is configured to handle multiple users, each with a separate account. Programming of device 205 permits each user to create an account that can store, test, display, and transmit data specific to any of the particular users. Device 205 does not contact any portion of the blood sample and is not contaminated from the blood sample. Each user is to use an individual reader 203. By plugging or coupling the individual reader 203 into device 205 and having the user log into his/her account, multiple users may share device 205.

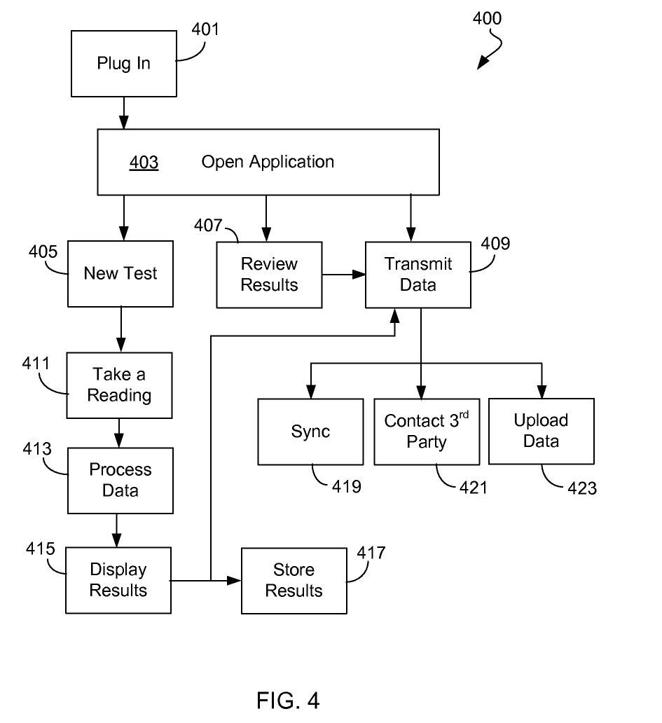

Referring now also to FIG. 4 in the drawings, a flowchart 400 illustrating the operation of system 101 is presented. Device 203 is placed in communication 401 with device 205. The insertion of testing strip 207 may be performed before or after communication between reader 203 and device 205 is achieved. Once communication is achieved, electronic power is provided to reader 203 and bi-directional communication is permitted.

A user is then able to access the programming or application 403 from device 205 in order to regulate the testing procedure. A user, after logining in to the particular user account, may select from at least one of three options: a new test 405, review historical results 407, and transmit data 409. Selecting a new test 405 permits power to be transferred to reader 203 along with command data to begin the testing process. Command data is received by reader 203 via engine 211 and other components. Reader 203 then interacts with the blood sample to develop a representative voltage. The voltage is transmitted 411 to device 205 for processing 413. Device 205 processes the voltage to determine the glucose reading. Other data may also be stored, for example the time of testing and location of the user. Results are then displayed 415 on display 221.

Results are automatically stored 417 on device 205. Results may be retained for any duration. Ideally, device 205 stores the data for a 6 month period of time but other times may be selected. At this time, results may also be automatically transmitted 409 to another device. Examples of types of transmission are an auto sync 419 with another electronic device, an auto upload 423 of information to the internet or a cloud-based application system, or auto transmission 421 to a third party individual. A third party individual may be any or all of a doctor, a family member of the user, a co-worker, and an emergency call center. The third party may be used to monitor levels from a remote location and to identify the need for medical assistance. For example, when a user performs a test and the glucose reading is below a prescribed safety level where the safety of the user is at risk, the third party may be able to send a notification or contact the user to assess the condition of the user. The third party may be able to contact emergency personnel and dispatch the emergency personnel to the user's location. In some embodiments, device 205 may automatically contact emergency personnel and/or provide the option to the user to contact emergency personnel by displaying a selected “button” for an emergency call.

Although automatic transmission of data has been described, it is understood that a user may elect to turn on or off the automatic feature of the transmission of data. It is also understood that a user has the ability to selectively choose the particular third party(s) and type of communication sent to each. Depending on the type of device that device 205 is, any one of a phone call, a text, an email, instant message, social media message, or fax may be used for communications to a third party. Other types of communication are understood as being possible and the list is in no way considered limiting.

Transmissions of data to third parties and through the syncing of systems or uploading data to the internet has been discussed. However, device 205 is configured to also provide alerts or notifications to the user. The alerts may act as a reminder for the user to take a test of their glucose reading or a warning that they failed to take a test at a prescribed time. Additionally, the alert may be given to the user following a test to bring the user's attention to a particular set of results or to perform an additional task. Each alert or notification may be used by a user to manage their glucose levels and interact with third parties. Notifications may be customizable to fit the user's need.

The reviewing of results 407 are automatically provided to a user following a particular test. Historical data is accessible at any time on device 205. Another feature of device 205 is the ability to automatically track and adjust to the specific time zone a user may be in. Device 205 is configured to adjust the times for tests taken to the selected time zone the user may be in. This auto adjust feature allows the user a more regulated schedule in testing. It is understood that once testing is completed, reader 203 is no longer necessary to be in communication with device 205. The transmission of data, providing of reminders or alerts, and the review of data may be done by a user with the device 205 alone. When the user is finished with the program, the program is closed and device 205 may be used for other services.

The current application has many advantages over the prior art including at least the following: (1) use of separate and distinct devices—one device to generate the representative voltage and a second device to process and store the results; (2) multi-user storage and processing device for testing; (3) ability to generate customizable alerts or notifications for a user or for a third party; (4) minimized and compact design for a reader; and (5) ability to auto adjust for time zones.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. An interactive glucometer system, comprising:
a test strip configured to receive a blood sample;
a glucose reader configured to interact with the test strip to generate a voltage representative of a glucose level within the blood sample, the glucose reader configured to transmit the voltage; and
an electronic device configured to receive and interpret the voltage from the glucose reader to generate a glucose reading, the electronic device configured to store and display data related to the glucose reading, the electronic device being independent and detachable from the glucose reader;
wherein the electronic device provides power and command data to the glucose reader so as to generate the voltage.

2. The interactive glucometer system of claim 1, wherein a user is able to manage, view, and transmit the data while disconnected from the glucose reader.

3. The interactive glucometer system of claim 1, wherein the data is stored on the electronic device independent from the glucose reader, so as to permit a user to view and transmit data in real time without communication from glucose reader.

4. The interactive glucometer system of claim 1, wherein the electronic device is at least one of a portable phone and an electronic tablet.

5. The interactive glucometer system of claim 1, wherein activation of the glucose reader is made from the electronic device.

6. The interactive glucometer system of claim 1, wherein the glucose reader includes a lancing device configured to prick a user in order to release a blood sample from the user.

7. The interactive glucometer system of claim 1, wherein the data is displayed on the electronic device in at least one of a graphical form, numerical form, and an audible form.

8. The interactive glucometer system of claim 1, wherein the electronic device is configured to automatically transmit the data to a third party following a test.

9. The interactive glucometer system of claim 8, wherein the third party is at least one of a doctor, a family member of the user, a co-worker, and an emergency call center.

10. The interactive glucometer system of claim 1, wherein the electronic device is configured to provide an alert notification to the user in accordance with the timing of a test.

11. The interactive glucometer system of claim 1, wherein the electronic device is configured to provide an alert notification to the user in accordance to test results.

12. The interactive glucometer system of claim 9, wherein transmission of the data is performed through wireless communications.

13. The interactive glucometer system of claim 1, wherein the glucose reader is detachable to allow for a secondary glucose reader to be used with the electronic device.

14. The interactive glucometer system of claim 1, wherein the electronic device is configured to monitor time across multiple time-zones.

15. The interactive glucometer system of claim 1, wherein the electronic device is configured to provide one or more reminders to a user.

16. A method of managing glucose levels, comprising:
applying a measure of blood to a test strip;
inserting the test strip into a glucose reader, the glucose reader housing measurement engine configured to interact with the blood and the test strip to produce a voltage representative to a level of glucose within the blood,
transmitting the voltage from the glucose reader to an independent electronic device, the electronic device configured to provide power and instructional data to the glucose reader, the electronic device receiving the voltage;
processing the voltage to generate a glucose reading; and
storing and displaying data related to the glucose reading.

17. The method of claim 16, further comprising:
reviewing the data associated with the glucose reading automatically within the electronic device; and

18. The method of claim 16, further comprising:
Automatically generating an alert notification to one of a user of the electronic device and a remote third party.

19. The method of claim 16, wherein the electronic device is configured to operate with multiple users, each user having a separate profile account on the electronic device, wherein each user uses a separate glucose reader to generate the voltage.

20. The method of claim 16, further comprising:
Setting an alert notification on the electronic device configured to convey a message to a user at a predefined time.
